(19) United States
(12) Patent Application Publication
(10) Pub. No.: US 2023/0380717 A1
(43) Pub. Date: Nov. 30, 2023

(54) RESPIRATION MONITORING SYSTEM USING A STRUCTURED LIGHT

(71) Applicant: GENTEX CORPORATION, Zeeland, MI (US)

(72) Inventor: Bar Mayo, Rehovot (IL)

(73) Assignee: GENTEX CORPORATION, ZEELAND, MI (US)

(21) Appl. No.: 18/203,126

(22) Filed: May 30, 2023

Related U.S. Application Data
(60) Provisional application No. 63/347,108, filed on May 31, 2022.

(51) Int. Cl. A61B 5/08; A61B 5/113; A61B 5/00

(52) U.S. Cl. CPC A61B 5/0816; A61B 5/746; A61B 5/742; A61B 5/113

(57) ABSTRACT

A monitoring system for measuring and monitoring a respiratory rate of a subject includes an optical projector configured to project an optical pattern and an image capturing module configured to capture images of the optical pattern reflecting from a surface. The monitoring system further comprises an alarm device, a processor, and a memory. The memory includes instructions that, when executed by the processor, cause the processor to monitor the respiration rate of the subject based on changes to the optical pattern reflected from the surface. The processor is further caused to determine if the monitored respiration rate is within a threshold variance and, upon a determination that the monitored respiration rate is outside of the threshold variance, generate a notification with the alarm device.

### CROSS-REFERENCE TO RELATED APPLICATION

This application claims priority to and the benefit under 35 U.S.C. § 119(e) of U.S. Provisional Application No. 63/347,108, filed on May 31, 2022, entitled “RESPIRATION MONITORING SYSTEM USING A STRUCTURED LIGHT,” the disclosure of which is hereby incorporated herein by reference in its entirety.

### FIELD OF THE DISCLOSURE

The present disclosure generally relates to a structured light monitoring system, and more particularly to utilizing a structured light to monitor breathing.

### SUMMARY OF THE DISCLOSURE

According to one aspect of the present disclosure, a monitoring system for measuring and monitoring a respiratory rate of a subject includes an optical projector configured to project an optical pattern and an image capturing module configured to capture images of the optical pattern reflecting from a surface. The monitoring system further comprises an alarm device, a processor, and a memory. The memory includes instructions that, when executed by the processor, cause the processor to monitor the respiration rate of the subject based on changes to the optical pattern reflected from the surface. The processor is further caused to determine if the monitored respiration rate is within a threshold variance and, upon a determination that the monitored respiration rate is outside of the threshold variance, generate a notification with the alarm device.

According to another aspect of the present disclosure, a monitoring system for measuring and monitoring a respiratory rate of a subject in a vehicle includes a structured light assembly. The structured light assembly includes an optical projector configured to project an optical pattern and an image capturing module configured to capture images of the optical pattern reflecting from a surface. The monitoring system further comprises an alarm device, a processor, and a memory. The memory includes instructions that, when executed by the processor, cause the processor to monitor the respiration rate of the subject based on changes to speckle content of at least a portion of the optical pattern reflecting from the surface. The processor is further caused to determine if the monitored respiration rate is within a threshold variance and, upon a determination that the monitored respiration rate is outside of the threshold variance, generate a notification with the alarm device.

According to yet another aspect of the present disclosure, a monitoring system for measuring and monitoring a respiratory rate of a subject includes an optical projector including a laser configured to project an optical pattern that includes at least one light spot and an image capturing module configured to capture images of the optical pattern reflecting from a surface. The monitoring system further comprises an alarm device, a processor, and a memory. The memory includes instructions that, when executed by the processor, cause the processor to monitor the respiration rate of the subject based on changes to the optical pattern reflected from the surface. The processor is further caused to determine if the monitored respiration rate is within a threshold variance and, upon a determination that the monitored respiration rate is outside of the threshold variance, generate a notification with the alarm device.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1A is a perspective view of an automobile that incorporates a structured light monitoring system, according to one aspect of the present disclosure;

FIG. 1B is a perspective view of an airplane that incorporates the structured light monitoring system, according to one aspect of the present disclosure;

FIG. 1C is a perspective view of a building that incorporates the structured light monitoring system, according to one aspect of the present disclosure;

FIG. 2 an interior view of a vehicle that incorporates the structured light monitoring system, according to one aspect of the present disclosure;

FIG. 3 schematically illustrates the structured light monitoring system, according to one aspect of the present disclosure;

FIG. 4 illustrates an image captured that includes a light spot reflected from a surface, according to one aspect of the present disclosure;

FIG. 5 illustrates an image captured that includes a light spot reflected from the surface after the surface has moved in a micro-scale, according to one aspect of the present disclosure;

FIG. 6 illustrates a monitoring system monitoring a respiration rate of an individual over a period of time, according to one aspect of the present disclosure;

FIG. 7 illustrates an image captured from an image capturing module of an individual, according to one aspect of the present disclosure;

FIG. 8 graphically illustrates an unfiltered breathing signal of the individual, according to one aspect of the present disclosure;

FIG. 9 graphically illustrates a frequency domain of the breathing signal, according to one aspect of the present disclosure;

FIG. 10 graphically illustrates a filtered breathing signal of the individual, according to one aspect of the present disclosure;

FIG. 11 schematically illustrates a control system of the structured light monitoring system, according to one aspect of the present disclosure; and

FIG. 12 is a method of monitoring the respiration rate of a subject with the structured light monitoring system, according to one aspect of the present disclosure.

### DETAILED DESCRIPTION

The present illustrated embodiments reside primarily in combinations of method steps and apparatus components related to a structured light monitoring system. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

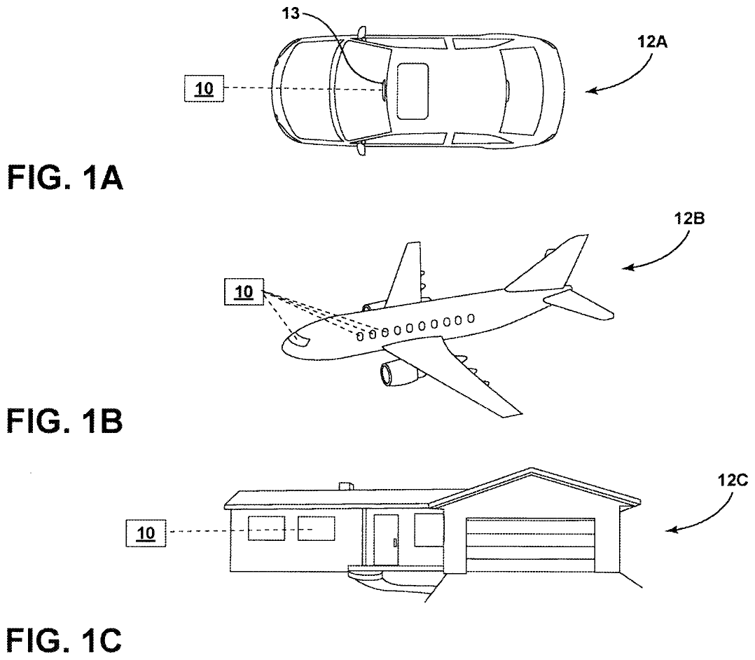

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to the surface of the device closer to an intended viewer of the device, and the term “rear” shall refer to the surface of the device further from the intended viewer of the device. However, it is to be understood that the disclosure may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

Referring to FIGS. 1A-1C, reference numeral 10 generally designates a monitoring system (e.g., a structure light monitoring system). The monitoring system 10 is configured to monitor the breathing of one or more individuals (i.e., persons or animals) within a variety of environments. More particularly, the monitoring system 10 may be incorporated with one or more structures 12A-12C. For example, FIG. 1A illustrates a vehicle 12A employing the monitoring system 10. All or some components of the monitoring system 10 may be located within an interior rearview mirror 13 and/or other interior vehicle locations that orient the monitoring system 10 into an interior cabin of the vehicle. The vehicle 12A may include a commercial vehicle, an emergency vehicle, a residential vehicle, a train, or the like. FIG. 1B illustrates an airplane 12B employing the monitoring system 10. The monitoring system 10 may be located within one or more areas to monitor occupants of the airplane 12B. For example, the monitoring system 10 may be located in a cockpit to monitor the status of a pilot. FIG. 1C illustrates a building 12C employing the monitoring system 10 to monitor one or more occupants within the building 12C. The building 12C may be a residential building, a commercial building, a medical facility, a retirement home, a nursery, a veterinary clinic, an animal facility, and/or the like. Generally speaking, the monitoring system 10 may be incorporated into any environment wherein breathing and/or other micro-movement behaviors of an individual can be monitored (e.g., a heartbeat, pulse, and/or the like).

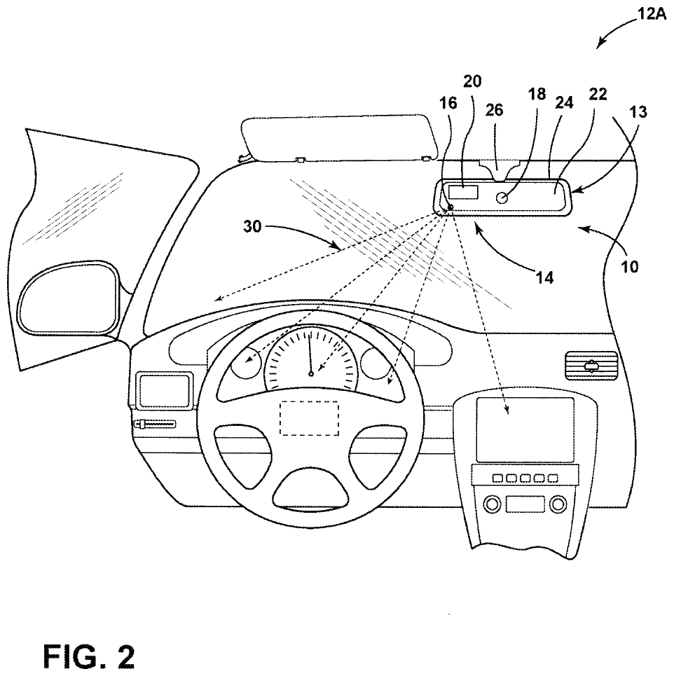

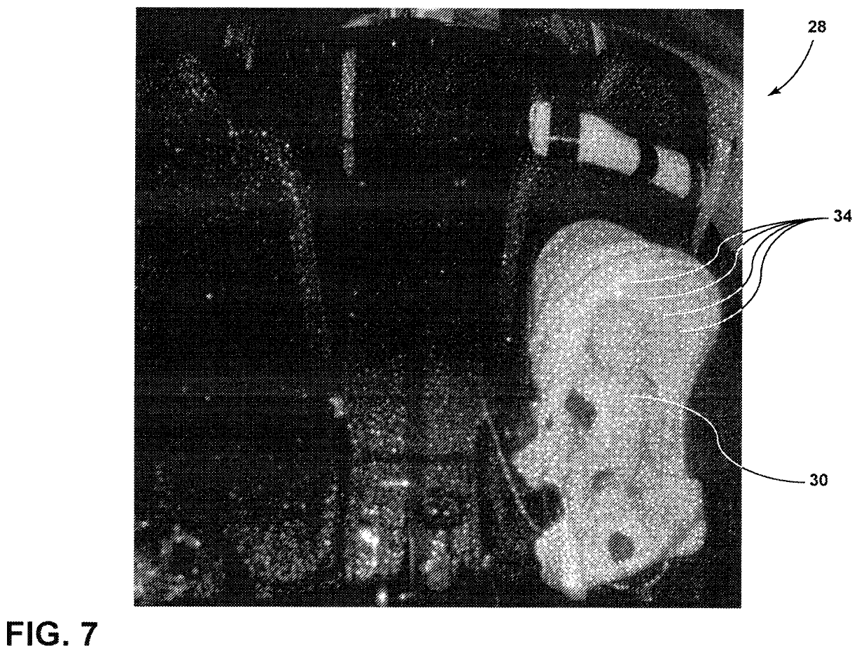

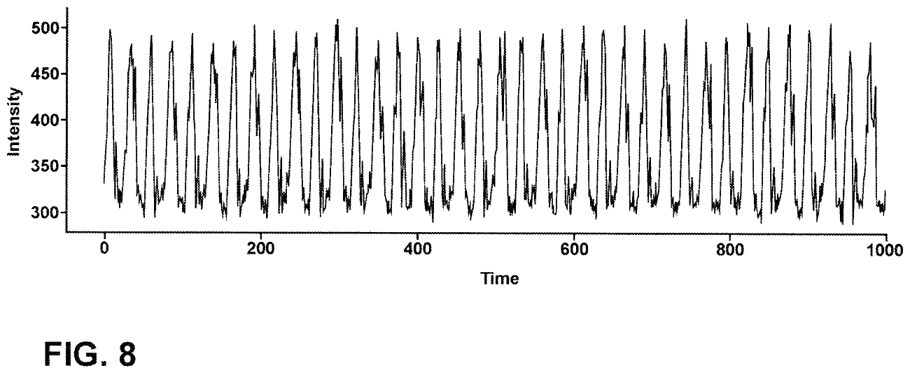

With reference now to FIG. 2, the monitoring system 10 includes a vision system, such as a structured light assembly 14. The structured light assembly 14 includes at least one optical projector 16 and at least one image capturing module 18. In some embodiments, the structured light assembly 14 (e.g., the optical projector 16 and/or the image capturing module 18) may be located within and/or otherwise coupled to the rearview mirror 13. The monitoring system 10 may further include an alarm device 20, for example, a display device located in and/or otherwise coupled to the interior rearview mirror 13, other interior vehicle locations, monitors, portable electronic devices, audio systems in the vehicle, localized audio devices, haptic feedback devices, and/or the like. The rearview mirror 13 may include a partially reflective, partially transmissive element 22, such as an electro-optic device that defines a viewing area of the rearview mirror 13. The rearview mirror 13 includes a housing 24 that may support the alarm device 20 and the partially reflective, partially transmissive element 22. The rearview mirror 13 may further include a mounting member 26 that couples the housing 24 to the interior cabin of the vehicle 12A. In some embodiments, the image capturing module 18 and the optical projector 16 are coupled to other areas of the vehicle 12A, such as with a dashboard, an overhead console, or another portions, such as the interior cabin. As will be described in greater detail below, the image capturing module 18 may be packaged (i.e., in a static relationship) with the optical projector 16. The image capturing module 18 is positioned to capture images 28 (FIG. 7) of the individuals within the vehicle 12A.

With continued reference to FIG. 2, the optical projector 16 may include one or more laser projectors. More particularly, the optical projector 16 may be configured as one or more single mode or multi-mode lasers operating within a wavelength spectrum, for example, the infrared (IR) spectrum. Accordingly, in some examples, the image capturing module 18 is a camera operable within the IR spectrum (or other spectrums corresponding to the optical projector 16). The image capturing module 18 may have a field of view that covers one or both of a front compartment and a rear compartment of the interior cabin. In this manner, the image capturing module 18 may be oriented to capture images 28 of a location of multiple or specific locations where an individual would be located.

With still continued reference to FIG. 2, the present disclosure may be used with a rearview mirror 13, such as that described in U.S. Pat. Nos. 9,505,349 and 10,739,591, which are hereby incorporated herein by reference in their entirety. The alarm device 20 may generate a notification including at least one of audible, visual, or haptic stimuli. For example, a visual notification may be generated on the rearview mirror 13 (e.g., the display) that includes a respiration rate, an audible notification may be generated that provides a beep sound or voice recommending checking a subject (e.g., in a medical environment), and a haptic notification may include shaking of a vehicle seat, steering wheel, personal electronic device, and/or the like.

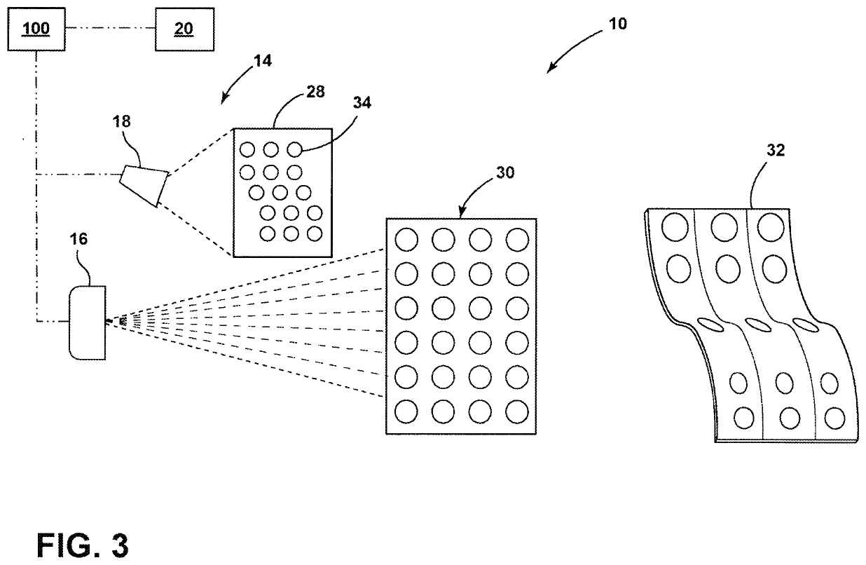

With reference now to FIG. 3, the optical projector 16 is configured to display a structured optical pattern 30 on an object 32 (e.g., an individual or something associated with an individual). The structured optical pattern 30 may include an array of shapes, such as spots 34, a series of alternating stripes of light, alternating intensity, and/or the like, that may be captured by the image capturing module 18. Regardless of the shape and distribution of the shapes (e.g., spots 34), when the surface of the object 32 reflecting the spots 34 moves, the spots 34 also move and this movement is captured by the image capturing module 18. Under a first mode of operation, a control system 100 may process the images 28 captured by the image capturing module 18 and extrapolate movement of the spots 34 into a depth of the surface based on the principles of triangulation and known geometries between image capturing module 18, the optical projector 16, and the distribution of structured optical pattern 30. For example, the control system 100 may be configured to determine movement based on an outer perimeter or a center of gravity of each spot 34. Under the first mode of operation, the image capturing module 18 and optical projector 16 may be closely and rigidly fixed on a common optical bench structure (e.g., within the rearview mirror 13 or other shared location) and, based on the known spacing between the image capturing module 18 and optical projector 16 and distribution of the structured optical pattern 30, the reflected spot 34 location can be captured along an epipolar line, which, in turn, can be triangulated to extract a depth of the surface. The depth of the surface at each spot 34 can then be used to extrapolate a contour of the surface. Likewise, changes in depth can be used to extrapolate the present location of the surface and movement of the surface as a function of time.

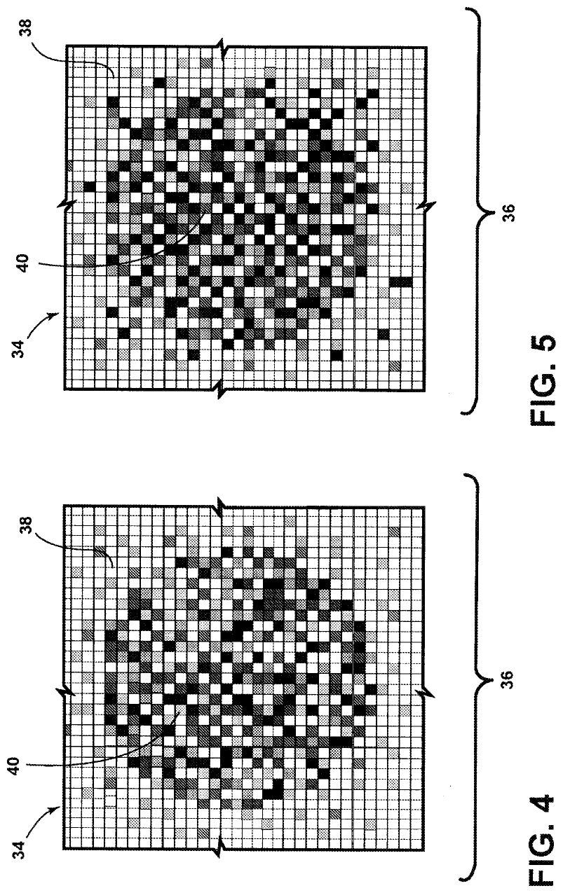

With reference now to FIGS. 4 and 5, the monitoring system 10 is also configured to operate under a second mode of operation. In the second mode of operation, a speckle content of each reflected spot 34 can be monitored to detect internal intensity distribution changes of the surface under the principles of speckle interferometry, such as tilting or other movements in a micro-scale (e.g., a micrometer or a micro-radian scale). For example, when a surface exhibits roughness, micro-radian changes in tilt affect the reflection of reflected spot 34 and, therefore, also the speckle content. The image capturing module 18 and the optical projector 16 may be spaced from one another in accordance with the second mode of operation. For example, the optical projector 16 and the image capturing module 18 may be located in different locations around the interior of the vehicle 12A. However, it should be appreciated that a single image capturing module 18 may be packaged with the optical projector 16 and utilized for both the first and second modes of operation.

With continued reference to FIGS. 4 and 5, the micro-scale detection can be utilized for monitoring a physiological condition of an occupant that may be difficult under the first mode of operation. For example, various vital signs of the occupant can be monitored by detecting micro motion of the occupant (e.g., the body surface), a covering (e.g., clothing or blanket) surface, a car seat surface, and a child seat surface. In this manner, even if spots 34 are not reflected directly from the body surface, micro-scale movements from the occupant are imparted on surfaces surrounding the occupant that can be detected, thus allowing occupant monitoring without a direct line of sight to the occupant's body. This can be particularly beneficial in scenarios where a child is in a rear-facing child car seat and/or covered by a blanket. More particularly, the speckle content of each reflected spot 34 is captured in the image 28 by the image capturing module 18.

FIG. 4 illustrates one of the images 28 of one of the reflected shapes that is projected onto a surface, and FIG. 5 illustrates one of the images 28 of the reflected spot 34 after the surface has changed position. It is contemplated that a timing between the image 28 captured in FIGS. 4 and 5 may be within one second, a millisecond, a microsecond, or any interval of time that allows for detection of small changes in position (e.g., micro-scale) of the surface. The speckle content in the image 28 of each spot 34 includes a pixel array 36 having corresponding values associated with each pixel. For example, the pixel array 36 may have pixel data that includes at least one value corresponding to a grayscale intensity value that corresponds to the level of reflected light toward the image capturing module 18. In general, under constant lighting conditions and no changes of position of the surface, the intensity values, or the pixel values, for each of the pixels in the pixel array 36, remain relatively constant or within a threshold range/profile of grayscale intensity. For example, because the structured light assembly 14 (e.g., via the control system 100) may be configured to capture micro-scale changes in position, some “noise” may be presented in the image 28 thereby affecting the pixel values. As will be described in greater detail below, the control system 100 may be configured to differentiate between the noise and actual movement of the object 32 in the interior of the vehicle 12A or other structure. For example, in some embodiments, the control system 100 may be configured to determine a baseline amount of noise (e.g., engine vibrations, road conditions, or other external factors that affect relative movement between the optical projector 16, the image capturing module 18, and the surface). This baseline may be determined by comparing and/or profiling changes in position of shapes (i.e., under the first mode of operation) or changes in speckle content of a reflected spot 34 (i.e., under the second mode of operation).

Still referring to FIGS. 4 and 5, the pixel array 36 may include a first portion 38 corresponding to an area surrounding the reflected spot 34 and a second portion 40 corresponding to the reflected spot 34. The second portion 40 may approximate the shape of the reflected spot 34, which, in this case, has a circular Gaussian shape. Minor changes, differentiated from any noise, are detected by the control system 100. For example, the minor change or alteration of pixel values, as a result of positional changes (e.g., micrometer scale) of the surface (e.g., FIGS. 3 and 4) may correspond to a redistribution of the black or dark pixels.

As explained previously, the minor positional changes of the surface may correspond to vital signs of an occupant. These vital signs may include the presence, rate and magnitude of breathing (i.e., the respiration rate), heartbeat, pulse, and/or other vital signs or physiological conditions of the occupant in the vehicle 12A. In general, the control system 100 may be configured to execute breathing, pulse, or other detection algorithms for determining vital signs or physiological conditions of the occupant (i.e., a person or an animal). Although not illustrated in detail, the control system 100 may evaluate some or all of the plurality of shapes projected by the optical projector 16 and amalgamate the image 28 corresponding with each of the plurality of shapes to further refine the determination of vital signs or physiological conditions of an occupant. For example, the control system 100 may employ one or more statistical modeling techniques to amalgamate or otherwise average the change in the pixel values of each reflected spot 34 in order to differentiate against noise and/or vibrations caused by movement of the vehicle 12A (or other structure 12B, 12C) from environmental or operational factors (e.g., gear shifting, braking, engine vibrations, road conditions, and/or the like).

It is further contemplated that the pixel data presented in FIGS. 4 and 5 may have a lower or higher resolution than the resolution depicted. For example, each reflected spot 34, may comprise any number of pixels corresponding to the particular resolution of the image capturing module 18. In general, the control system 100 may employ the principles of speckle interferometry in the second mode of operation, and thus evaluate the structured optical pattern 30 based on changes to the speckle content of each of the plurality of shapes (e.g., spots 34). The second mode of operation is performed on a spot-by-spot basis of the individual reflections of the shapes.

The image capturing module 18 captures the reflected spots 34, and each spot 34 may be associated with a pixel count and a perceived intensity value. The perceived intensity value in each pixel depends on the interference of the microstructure of the object 32 illuminated and the projected light where the structured optical pattern 30 is diffusely reflected as a reflected spot 34 (FIG. 5). The illuminated and projected light from the object 32 can, therefore, be used to determine characteristics of the object 32 (e.g., shape, position, depth, location, etc.) and any changes to the object 32 (e.g., shape, position, micro-movements, etc.). For example, when the object 32 (i.e., the individual or occupant) moves (e.g., tilts relative to the at least one image capturing module 18 and/or the at least one optical projector 16), a change in the reflected spot 34 will result. This change is very sensitive, so a micro-radian scale tilt produces a detectable change in the reflected spot 34 that can be captured by the at least one image capturing module 18. The reflected light in the reflected spot 34 is dependent on characteristics of the light projected in the structured optical pattern 30 (wavelength and intensity) and the relative locations of at least one optical projector 16, the object 32 (e.g., material reflectivity and textures), and the image capturing module 18. In operation, all of the relative positions are substantially constant except for the location of the object 32, movement of which can be determined in a micro-meter scale. Changes in the location of the object 32 in the micro-meter scale (i.e., micro-vibrations) typically will not result in changes in the shape of the structured optical pattern 30 but rather a perceived intensity value of the pixels of each spot in the reflected spot 34.

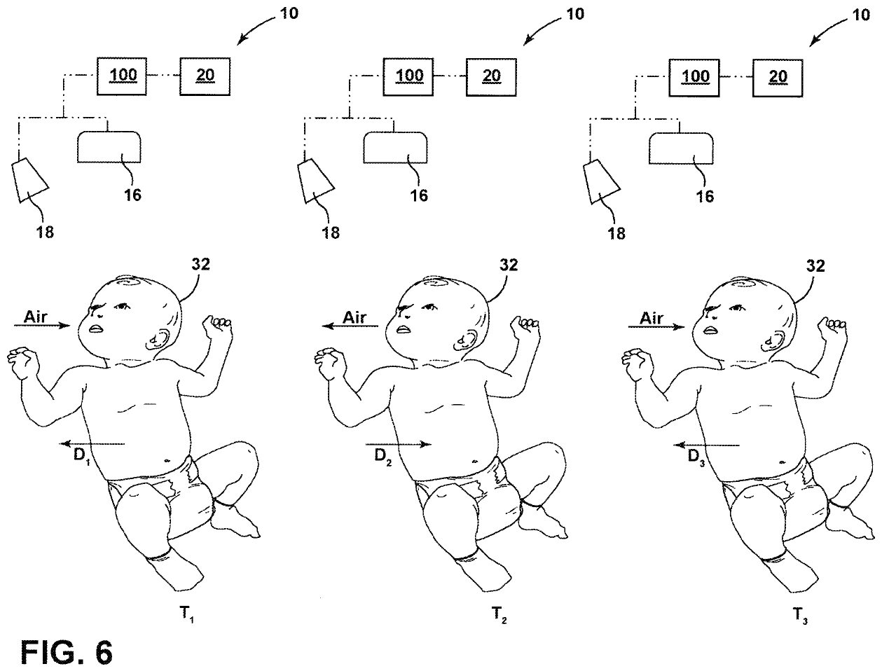

As best illustrated in FIG. 6, these changes to the object 32 can be modeled for certain activities, such as breathing. For example, a healthy adult typically has a normal respiration rate of 12 to 20 breaths per minute, whereas a healthy infant typically has a normal respiration rate of 40 to 60 breaths per minute. A semi-linear progression can be found for children between infancy and adulthood where normal respiration rates typically decrease as children age. Importantly, these healthy respiration rates are known within the medical community and can be relied upon as accurate. Likewise, other vital signs or physiological condition progressions are known by the medical community and may be utilized as a variable or baseline by the control system 100 to monitor the individual based on the age, size, sex, and species of the individual. Accordingly, the object 32 may be a person (e.g., an infant) and the monitoring system 10 may be configured to recognize respiration rates and monitor for normalcy within expected ranges. More particularly, in situations where monitoring a respiratory rate of an infant is beneficial, slight respiratory movements of the infant, such as the chest, head, and other body portions, can be monitored by filtering out other types of movements. For example, when the monitoring system 10 is implemented in a building 12C, non-respiratory related movement may include rolling, adjusting, arm and leg movements, adjustments in clothing, etc. In addition, when the monitoring system 10 is implemented in a vehicle 12A or airplane 12B, other types of non-respiratory related movement may additionally be filtered, such as turbulence, road conditions, acceleration of the vehicle 12A or airplane 12B, etc. FIG. 7 illustrates the reflected spot 34 on an object 32 that includes an infant and a child seat, where the perceived intensity value of the pixels (and changes thereto) can be determined with image data received (e.g., images 28) from the at least one image capturing module 18.

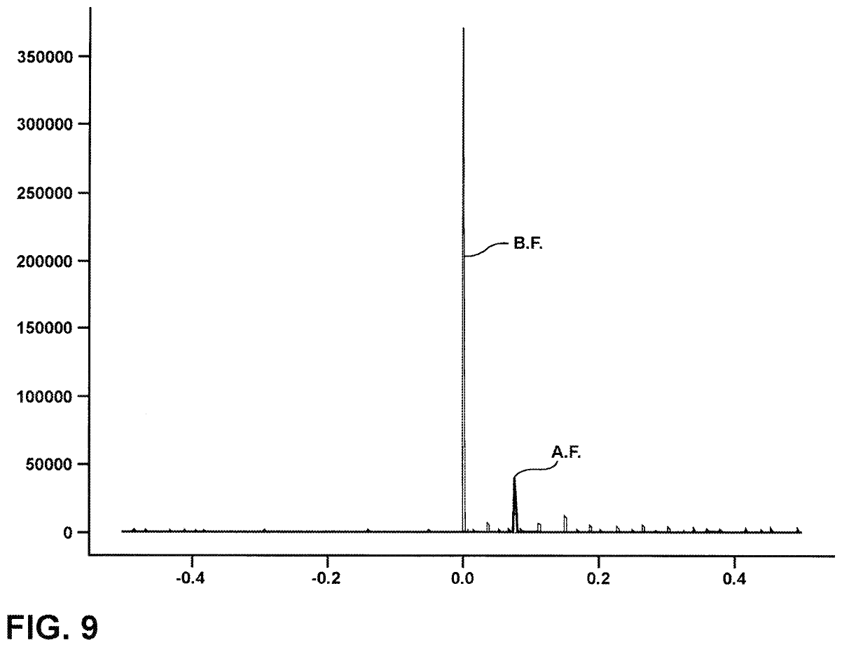

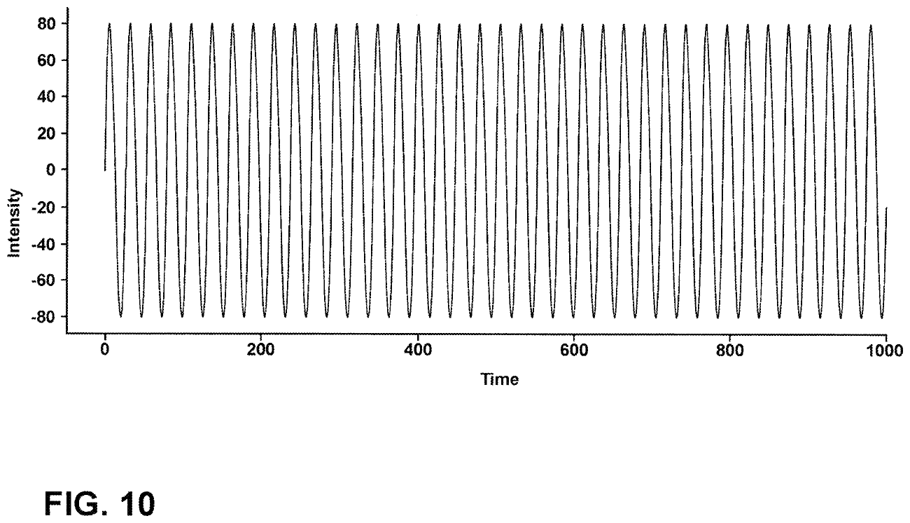

With reference now to FIGS. 8-10, the term “filtering” as used herein may refer to one or more breathing models that accurately reflects frequency and/or amplitude of movement caused by breathing and it may also refer to discarding other types of captured movements that do not relate to breathing (i.e., non-respiratory related movement). In some embodiments, the breathing models are pre-saved in the control system 100 prior to monitoring and categorized by age, weight, sex, heath status, etc. In some embodiments, the monitoring system 10 develops a baseline frequency/amplitude for an individual as a first step in monitoring, and the baseline frequency/amplitude can then be saved into the control system 100. In other embodiments, the monitoring system 10 incorporates both pre-saved models and baseline frequency/amplitudes during monitoring that can be compared by the control system 100. FIG. 8 illustrates a breathing signal with the changes in the perceived intensity value in a single pixel in one of the spots in the reflected spot 34 represented over a period of time where the object 32 is an infant's stomach area, where the Y value is equal to a spot's pixel value, and the x value is equal to a frame number. FIG. 9 illustrates the frequency domain from FIG. 8 before filtering “B.F.” and after filtering “A.F.” where the Y value is equal to amplitude and the x value is equal to frequency. FIG. 10 is a filtered representation of the breathing signal illustrated in FIG. 7, where the Y value is equal to a spot's 34 pixel value and the x value is equal to a frame number. In some embodiments, there may be changes to the perceived intensity value as a result of the filtering. These changes in perceived intensity can be scaled to an accurate representation during monitoring.

With reference back to FIG. 6, the baseline frequency/amplitudes may be determined over a period of time T₁-T_{N}, for example, 5 minutes or less, 1 minute or less, 45 seconds or less, 30 seconds or less, or 15 seconds or more. As an individual breathes, movements D1-D3 (e.g., micro-movements) between inhaling D2 and exhaling D1 and D3 are measured to determine an amplitude. The amplitude may vary based on clothing, subject position, etc. Throughout the period of time T₁-T_{N} (e.g., T₁-T₃), a number of breaths can be determined based on movement D between inhaling D2 and exhaling D1 and D3. The total number of breaths throughout the period of time T₁-T_{N} can be used to determine a respiration rate. The measured respiration rate may then be compared to the pre-saved models to ensure accuracy. In some embodiments, a hybrid model may be saved that incorporates features of the pre-saved model and the baseline frequency/amplitudes, for example, an average between both.

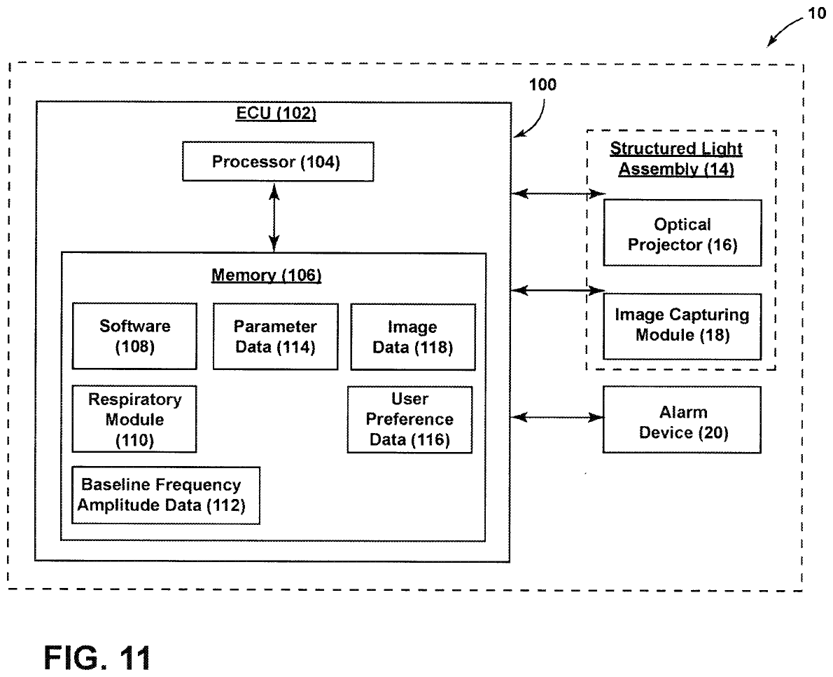

FIG. 11 schematically illustrates the control system 100 of the monitoring system 10. The control system 100 may include an electronic control unit (ECU) 102. The ECU 102 may include a processor 104 and a memory 106. The processor 104 may include any suitable processor 104. Additionally, or alternatively, the ECU 102 may include any suitable number of processors, in addition to or other than the processor 104. The memory 106 may comprise a single disk or a plurality of disks (e.g., hard drives) and includes a storage management module that manages one or more partitions within the memory 106. In some embodiments, memory 106 may include flash memory, semiconductor (solid state) memory, or the like. The memory 106 may include Random Access Memory (RAM), a Read-Only Memory (ROM), Electrically Erasable Programmable Read-Only Memory (EEPROM), or a combination thereof. The memory 106 may include instructions that, when executed by the processor 104, cause the processor 104 to, at least, perform the functions associated with the components of the monitoring system 10. The at least one optical projector 16, the at least one image capturing module 18, and the alarm device 20 may therefore be controlled by the ECU 102. The memory 106 may therefore include software 108, pre-saved respiratory models 110, parameter data 112, baseline frequency/amplitude data 114 captured by the structured light assembly 14, user preference data 116, and image data 118 (e.g., a series of images 28).

With continued reference to FIG. 11, the software 108 may be updated to incorporate additional functionalities or override the functionalities as described herein. The pre-saved respiratory models 110 may include respiration rates based on age, sex, weight, health status, and/or other medical information related to a subject. The parameter data 112 may include predetermined thresholds wherein deviations between the amplitude and/or frequency of measured respiration rates generate a notification with the alarm device 20. The predetermined threshold of the parameter data 112 may vary based on which of the pre-saved respiratory models 110 is selected, for example, variances in respiration rates may be more or less common for medical conditions, age, health, weight, sex, etc. The baseline frequency/amplitude data 114 may include a detected respiration rate. The predetermined threshold of the parameter data 112 may also vary based on the baseline frequency/amplitude data 114, for example, individual respiration rates may vary within expected ranges. A healthy individual may have a slower respiration rate than average. As such, the predetermined threshold may measure variances from the baseline frequency/amplitude data 114 or an average of the baseline frequency/amplitude data 114 and the pre-saved respiratory models 110. The user preference data 116 may include user choices of the functionalities and/or order of functionalities. For example, a user interface on a full display mirror or other electronic device may allow a user to turn the monitoring system on and off, change the type of notification generated by the alarm device 20, etc. The image data 118 may include real-time images 28 where measured respiration rates of the subject that can then be compared to the pre-saved respiratory models 110 and/or the baseline frequency/amplitude data 114 for variances outside of a threshold provided by the parameter data 112.

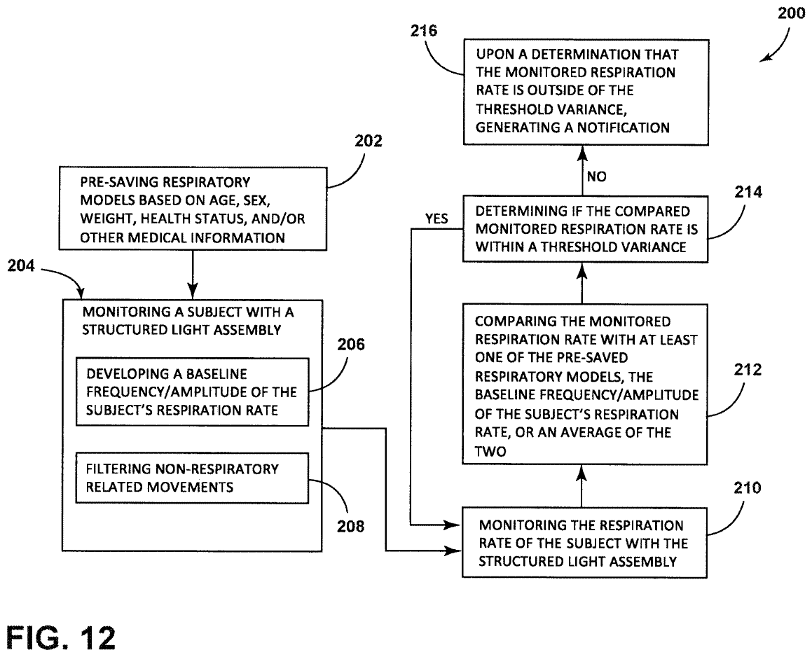

FIG. 12 illustrates a method 200 of monitoring a respiratory rate of a subject. At 202, the method 200 includes pre-saving respiratory models based on age, sex, weight, health status, and/or other medical information. At 204, the method 200 includes monitoring the subject with a structured light assembly. Step 204 may further include, at 206, developing a baseline frequency/amplitude of the subject's respiration rate. Step 204 may further include yet, at 208, filtering non-respiratory related movements. At 210, the method 200 includes monitoring the respiration rate of the subject with a structured light assembly. At 212, the method 200 may include comparing the monitored respiration rate with at least one of the pre-saved respiratory models, the baseline frequency/amplitude of the subject's respiration rate, or an average of the two. At 214, the method 200 includes determining if the compared monitored respiration rate is within a threshold variance. At 216, the method includes, upon a determination that the monitored respiration rate is outside of the threshold variance, generating a notification. If the monitored respiration rate is within the threshold variance, the method repeats steps 210 through 214. Steps of the method 200 may be saved in memory 106 and facilitated by the processor 104.

The invention disclosed herein is further summarized in the following paragraphs and is further characterized by combinations of any and all of the various aspects described therein.

According to one aspect of the present disclosure, a monitoring system for measuring and monitoring a respiratory rate of a subject includes an optical projector configured to project an optical pattern and an image capturing module configured to capture images of the optical pattern reflecting from a surface. The monitoring system further comprises an alarm device, a processor, and a memory. The memory includes instructions that, when executed by the processor, cause the processor to monitor the respiration rate of the subject based on changes to the optical pattern reflected from the surface. The processor is further caused to determine if the monitored respiration rate is within a threshold variance and, upon a determination that the monitored respiration rate is outside of the threshold variance, generate a notification with the alarm device.

According to another aspect, the alarm device is configured to generate at least one of an audible notification, a haptic notification, or a visual notification.

According to yet another aspect, the alarm device includes a display device.

According to still yet another aspect, the display device is located in a rearview mirror of a vehicle.

According to another aspect, an image capturing module is located in a rearview mirror.

According to yet another aspect, a rearview mirror includes a partially reflective, partially transmissive element.

According to still yet another aspect, an optical projector is configured to project an optical pattern within an infrared wavelength.

According to another aspect, an optical pattern includes at least one spot.

According to yet another aspect, a memory includes instructions that cause a processor to detect changes in a speckle content of an at least one spot.

According to still yet another aspect, a memory includes instructions that cause a processor to, based on a detected change of a speckle content, extrapolate micro-movements of a subject related to respiration.

According to another aspect, a memory includes instructions that cause a processor to develop a baseline characteristic of a subject's respiration rate and compare a monitored respiration rate with at least one of a plurality of pre-saved respiratory models, the baseline characteristic of the subject's respiration rate, or an average of the plurality of pre-saved respiratory models and the baseline characteristic of the subject's respiration rate.

According to yet another aspect, a baseline characteristic of a subject's respiration rate includes at least one of a frequency or an amplitude of respiration.

According to still yet another aspect, a memory includes instructions that cause a processor to filter non-respiratory related movements when developing a baseline characteristic of a subject's respiration rate.

According to another aspect of the present disclosure, a monitoring system for measuring and monitoring a respiratory rate of a subject in a vehicle includes a structured light assembly. The structured light assembly includes an optical projector configured to project an optical pattern and an image capturing module configured to capture images of the optical pattern reflecting from a surface. The monitoring system further comprises an alarm device, a processor, and a memory. The memory includes instructions that, when executed by the processor, cause the processor to monitor the respiration rate of the subject based on changes to speckle content of at least a portion of the optical pattern reflecting from the surface. The processor is further caused to determine if the monitored respiration rate is within a threshold variance and, upon a determination that the monitored respiration rate is outside of the threshold variance, generate a notification with the alarm device.

According to another aspect, changes in speckle content relate to movements in a micro-meter scale.

According to yet another aspect, the surface is a back of a child car seat.

According to still yet another aspect, an alarm device includes a display located in a rearview mirror assembly,

According to yet another aspect of the present disclosure, a monitoring system for measuring and monitoring a respiratory rate of a subject includes an optical projector including a laser configured to project an optical pattern that includes at least one light spot and an image capturing module configured to capture images of the optical pattern reflecting from a surface. The monitoring system further comprises an alarm device, a processor, and a memory. The memory includes instructions that, when executed by the processor, cause the processor to monitor the respiration rate of the subject based on changes to the optical pattern reflected from the surface. The processor is further caused to determine if the monitored respiration rate is within a threshold variance and, upon a determination that the monitored respiration rate is outside of the threshold variance, generate a notification with the alarm device.

According to another aspect, a laser is configured to project an optical pattern in an infrared wavelength of light and an image capturing module is configured to capture images within the infrared wavelength of light.

According to yet another aspect, a memory includes instructions that cause a processor to filter non-respiratory related movements when developing a baseline characteristic of a subject's respiration rate.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be appreciated that embodiments of the disclosure described herein may be comprised of one or more conventional processors and unique stored program instructions that control one or more processors to implement, in conjunction with certain non-processor circuits, some, most, or all of the functions of a monitoring system, as described herein. The non-processor circuits may include, but are not limited to, signal drivers, clock circuits, power source circuits, and/or user input devices. As such, these functions may be interpreted as steps of a method used in using or constructing a classification system. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Thus, the methods and means for these functions have been described herein. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

It will be understood that any described processes or steps within described processes may be combined with other disclosed processes or steps to form structures within the scope of the present disclosure. The exemplary structures and processes disclosed herein are for illustrative purposes and are not to be construed as limiting.

It is also to be understood that variations and modifications can be made on the aforementioned structures and methods without departing from the concepts of the present disclosure, and further it is to be understood that such concepts are intended to be covered by the following claims unless these claims by their language expressly state otherwise.

What is claimed is:

1. A monitoring system for measuring and monitoring a respiratory rate of a subject, the monitoring system comprising:
an optical projector configured to project an optical pattern and an image capturing module configured to capture images of the optical pattern reflecting from a surface;
an alarm device; and
a processor and a memory including instructions that, when executed by the processor, cause the processor to:
monitor a respiration rate of the subject based on changes to the optical pattern reflecting from the surface;
determine if the monitored respiration rate is within a threshold variance; and
upon a determination that the monitored respiration rate is outside of the threshold variance, generate a notification with the alarm device.

2. The monitoring system of claim 1, wherein the alarm device is configured to generate at least one of an audible notification, a haptic notification, or a visual notification.

3. The monitoring system of claim 2, wherein the alarm device includes a display device.

4. The monitoring system of claim 3, wherein the display device is located in a rearview mirror of a vehicle.

5. The monitoring system of claim 4, wherein the image capturing module is located in the rearview mirror.

6. The monitoring system of claim 5, wherein the rearview mirror includes a partially reflective, partially transmissive element.

7. The monitoring system of claim 1, wherein the optical projector is configured to project the optical pattern within an infrared wavelength.

8. The monitoring system of claim 1, wherein the optical pattern includes at least one spot.

9. The monitoring system of claim 8, wherein the memory further includes instructions that cause the processor to:
detect changes in a speckle content of the at least one spot.

10. The monitoring system of claim 9, wherein the memory further includes instructions that cause the processor to:
based on the detected changes of the speckle content, extrapolate micro-movements of the subject related to respiration.

11. The monitoring system of claim 1, wherein the memory further includes instructions that cause the processor to:
develop a baseline characteristic of the subject's respiration rate; and
compare the monitored respiration rate with at least one of a plurality of pre-saved respiratory models, the baseline characteristic of the subject's respiration rate, or an average of the plurality of pre-saved respiratory models and the baseline characteristic of the subject's respiration rate.

12. The monitoring system of claim 11, wherein the baseline characteristic of the subject's respiration rate includes at least one of a frequency or an amplitude of respiration.

13. The monitoring system of claim 11, wherein the memory further includes instructions that cause the processor to:
filter non-respiratory related movements when developing the baseline characteristic of the subject's respiration rate.

14. A monitoring system for measuring and monitoring a respiratory rate of a subject in a vehicle, the monitoring system comprising:
a structured light assembly including an optical projector configured to project an optical pattern and an image capturing module configured to capture images of the optical pattern reflecting from a surface;
an alarm device; and
a processor and a memory including instructions that, when executed by the processor, cause the processor to:
monitor a respiration rate of the subject based on changes to a speckle content of at least a portion of the optical pattern reflecting from the surface;
determine if the monitored respiration rate is within a threshold variance; and
upon a determination that the monitored respiration rate is outside of the threshold variance, generate a notification with the alarm device.

15. The monitoring system of claim 14, wherein the changes in the speckle content relate to movements in a micro-meter scale.

16. The monitoring system of claim 15, wherein the surface is a back of a child car seat.

17. The monitoring system of claim 14, wherein the alarm device includes a display located in a rearview mirror assembly.

18. A monitoring system for measuring and monitoring a respiratory rate of a subject, the monitoring system comprising:
an optical projector including a laser configured to project an optical pattern that includes at least one spot and an image capturing module configured to capture images of the optical pattern reflecting from a surface;
an alarm device; and
a processor and a memory including instructions that, when executed by the processor, cause the processor to:
monitor a respiration rate of the subject based on changes to the optical pattern reflecting from the surface;
determine if the monitored respiration rate is within a threshold variance; and
upon a determination that the monitored respiration rate is outside of the threshold variance, generate a notification with the alarm device.

19. The monitoring system of claim 18, wherein the laser is configured to project the optical pattern in an infrared wavelength of light and the image capturing module is configured to capture images within the infrared wavelength of light.

20. The monitoring system of claim 18, wherein the memory further includes instructions that cause the processor to:
filter non-respiratory related movements when developing a baseline characteristic of the subject's respiration rate.
